(19) United States
(12) Patent Application Publication
(10) Pub. No.: US 2019/0388389 A1
(43) Pub. Date: Dec. 26, 2019

(54) METHODS AND COMPOSITIONS OF CARNOSOL FOR INHIBITING UVB-INDUCED SKIN DAMAGE

(71) Applicant: Ohio University, Athens, OH (US)

(72) Inventors: Shiyong Wu, Athens, OH (US); Lingying Tong, Athens, OH (US)

(73) Assignee: Ohio University, Athens, OH (US)

(21) Appl. No.: 16/482,015

(22) PCT Filed: Jan. 31, 2018
(86) PCT No.: PCT/US18/16107

§ 371 Date: Jul. 30, 2019

Related U.S. Application Data
(60) Provisional application No. 62/456,269, filed on Feb. 8, 2017.

(51) Int. Cl. A61K 31/366; A61K 8/49; A61K 9/00; A61Q 19/00

(52) U.S. Cl. CPC A61K 31/366; A61K 8/498; A61K 2800/522; A61Q 19/004; A61K 9/0014

(57) ABSTRACT

Compositions and methods for inhibition UVB-irradiation damage by administering an effective amount of carnosol are described.

### RELATED APPLICATIONS

This application claims priority to U.S. Ser. No. 62/456,269 filed under 35 U.S.C. § 111(b) on Feb. 8, 2017, the disclosure of which is incorporated herein by reference in its entirety.

### STATEMENT REGARDING FEDERALLY SPONSORED RESEARCH

This invention was made with government support under Grant No. CA086928 awarded by the National Institutes of Health. The government has certain rights in the invention.

### SEQUENCE LISTING

The instant application contains a Sequence Listing which has been submitted via EFS-web and is hereby incorporated by reference in its entirety. The ASCII copy, created on Jan. 22, 2018, is named 3834_574061_SEQ_LIST_OU16009.txt, and is 532 bytes in size.

### BACKGROUND OF THE INVENTION

Ultraviolet B light (UVB) is a well-known carcinogen for skin cancer development and progression. Overexposure to UVB radiation leads to various skin cancers, including basal cell carcinoma (BCC), squamous cell carcinoma (SCC), and cutaneous malignant melanoma. One of the possible carcinogen mechanisms is that UVB radiation causes DNA damage through the induction of free radicals and reactive oxygen species (ROS).

### SUMMARY OF THE INVENTION

Described herein is the use of carnosol to reduce the UVB-induced intracellular ROS elevation, and use as a therapeutic for chemoprevention and treatment of skin cancers

In one aspect, there is a composition comprising an effective amount of carnosol as an active ingredient to reduce UVB-induced reactive oxygen species (ROS) levels in a cell, and to protect the cell from UVB-caused DNA damage; and, a pharmaceutically acceptable excipient, diluent, or carrier.

In certain embodiments, the composition includes carnosol present in an amount effective to inhibit DNA damage caused by exposure to UVB irradiation.

In certain embodiments, the composition includes carnosol present in an amount effective to inhibit skin cancer cell growth.

In certain embodiments, the composition includes carnosol present in an amount effective to protect the cell from UVB-induced transformation.

In certain embodiments, the composition includes carnosol present in an amount effective to decrease cancer cell progression.

In certain embodiments, the composition is a cosmetic or dermatological composition for coating skin cells of a subject.

In certain embodiments, the composition is in the form of a spray, mist, aerosol, lotion, cream, solution, oil, gel, ointment, paste, emulsion or suspension.

In another aspect, there is provided a method of treating a cell, comprising: administering an effective amount of carnosol to reduce UVB-induced reactive oxygen species (ROS) levels in the cell, and to protect the cell from UVB-caused DNA damage.

In certain embodiments, the carnosol is administered in an amount effective to effective to inhibit DNA damage caused by exposure to UVB irradiation.

In certain embodiments, the carnosol is present in an amount effective to inhibit skin cancer cell growth.

In certain embodiments, the carnosol is present in an amount effective to protect the cell from UVB-induced transformation.

In certain embodiments, the carnosol is present in an amount effective to decrease cancer cell progression.

In certain embodiments, the method comprises topically applying carnosol to the cell.

In certain embodiments, the cell is a skin cell. In certain embodiments, the the cancer cell is a squamous carcinoma cell.

Various objects and advantages of this invention will become apparent to those skilled in the art from the following detailed description of the preferred embodiment, when read in light of the accompanying drawings.

### BRIEF DESCRIPTION OF THE DRAWING

The patent or application file may contain one or more drawings executed in color and/or one or more photographs. Copies of this patent or patent application publication with color drawing(s) and/or photograph(s) can be provided by the U.S. Patent and Trademark Office upon request and payment of the necessary fees.

FIG. 1A: The chemical structure of carnosol.

FIG. 1B: Model for the effect of carnosol upon UVB radiation.

FIG. 2: Dose dependent effect of carnosol on UVB-induced intracellular ROS level. HaCaT cells were seeded in 96 well plate and incubated with CM-H₂DCFDA dye one hour prior to UVB exposure, with or without carnosol treatment. ROS was measured at 6 h post UVB radiation with indicated concentration of carnosol treatment using ex490/em520 nm. The data represents three to five sets of independent experiments. *p<0.05 versus corresponded control group, **p<0.05 versus UV alone.

FIGS. 3A-3B: Time dependent effect of carnosol on UVB-induced intracellular ROS level. HaCaT cells were seeded in 96 well plate and CM-H₂DCFDA dye was used to measure intracellular ROS level. Carnosol (20 μM) was added to cells 1 h prior to UVB exposure, and ROS level was measured at 20 min intervals post UVB. (FIG. 3A) Intracellular ROS level at 20 min intervals with or without carnosol treatment. (FIG. 3B) Statistical analysis of intracellular ROS level at 240, 480, 720 min post UVB radiation with or without carnosol (20 μM) treatment. *p<0.05 versus corresponded control group, **p<0.05 versus corresponded UV group.

FIGS. 4A-4B: Quantitative analysis of NO* and ONOO⁻ after UVB radiation. HaCaT cells were irradiated with UVB radiation in the presence or absence of carnosol (20 μM). At indicated time point, cells were stained with DAF-2DA or DAR. The fluorescent intensity was then measured and normalized to cell viability. (FIG. 4A) Quantitative measurement of NO*. (FIG. 4B) Quantitative measurement of ONOO⁻. *p<0.05 versus corresponded group with carnosol treatment upon UVB radiation.

FIGS. 5A-5B: Carnosol protects UVB-induced DNA damage. (FIG. 5A) HaCaT cells and (FIG. 5B) MEF cells were exposed to 50 mJ/cm² UVB radiation in the presence or absence of carnosol. Cells were lysed at indicated time point and protein levels of phosphorylated H2AX and Chk were measured. The data represents three sets of independent experiments.

FIG. 5C: Immunostaining of p-Chk in HaCaT cells upon UVB radiation with or without carnosol treatment. P-Chk was stained with green fluorescent, while nucleus was stained by DAPI. Scale indicates 20 μm.

FIG. 5D: Comet assay for HaCaT exposed to UVB radiation. After cell treatment, cells were trypsinized and collect for comet assay. The tail intensity was semi-quantitatively analyzed using Image J.

FIG. 5E: Quantitative measurement of cyclobutane pyrimidine dimer (CPD) formation, with and without UVB, and with and without carnosol. *p<0.05 versus corresponded control group, **p<0.05 versus corresponded UV group.

FIG. 5F: Quantitative measurement of 6-4 photoproducts (6-4PP), with and without UVB, and with and without carnosol. *p<0.05 versus corresponded control group

FIGS. 6A-6C: Carnosol protects UVB-induced cell death. Cells were exposed to UVB radiation with different dose of carnosol treatment. At indicated time point, cells were collected and Annexin V/PI apoptosis detection kit was used to detect cell apoptosis. (FIG. 6A) HaCaT cell survival rate with different concentrations (μM) of carnosol treatment at 12 h post UVB radiation. (FIG. 6B) Time dependent assay (h) in the presence or absence of carnosol treatment in HaCaT cells. (FIG. 6C) MEF cells treated with 20 μM carnosol at indicated time point (h). The error bars present the standard deviation of three sets of independent experiments. *p<0.05 versus control group; **p<0.05 versus corresponded UV group.

FIGS. 7A-7F: Carnosol inhibited UVB-induced NF-κB activation. Cells were exposed to UVB radiation in the presence or absence of carnosol. Cells were lysed at indicated time point and protein levels were measured by western blot analysis. (FIG. 7A) Western blot for IκB protein level in HaCaT cells at 2, 4 h post UVB radiation at 1, 10 or 20 μM carnosol treatment. (FIG. 7B) Western blot analysis for IκB protein level in HaCaT cells at 6 h post UVB radiation in the presence or absence of carnosol. (FIG. 7C) Western blot analysis for IκB protein level in MEF cells at 6 h post UVB radiation in the presence or absence of carnosol. (FIG. 7D) Western blot for 5276 site phosphorylation of NF-κB in HaCaT cells at 6 h post UVB with indicated carnosol concentration. (FIG. 7E) EMSA assay for HaCaT cells at 2, 4, 6 h post UVB radiation in the presence or absence of carnosol (20 μM). (FIG. 7F) Statistical analysis of the EMSA assay using two to three sets of independent experiments. *p<0.05 versus control group, **p<0.05 versus UV group at indicated time point.

FIG. 8: Carnosol decreases HaCaT transformation rate upon UVB radiation. HaCaT cells were exposed to 10 mJ/cm² UVB radiation every 48 h for 14 days. Cells were then collected and equal amount of cells were then re-seeded into 96 well plate with soft agar. After 10 days of incubation, cells were stained and dissolved and the fluorescent intensity was determined. *p<0.05 versus control group, **p<0.05 versus UV group.

FIGS. 9A-9B. Carnosol decreases A431 cancer cell progression. Squamous carcinoma cell A431 was used to determine the effect of carnosol on cancer cell progression. (FIG. 9A) Effect of carnosol on cell viability. Cells were treated upon 50 mJ/cm² UVB radiation with or without carnosol treatment. After 24 h, cells were collect and MTT assay was performed to determine the cell viability. *p<0.05 versus control group, **p<0.05 versus UV group at indicated time point. (FIG. 9B) Effect of carnosol on clonogenic assay. Cells were exposed to 8 mJ/cm² UVB exposure in the presence or absence of carnosol. 5×10³ cells were plated into 6 well plate immediately after treatment and incubated for 8 days. The error bars present the standard deviation of three sets of independent experiments. *p<0.05 versus control group, **p<0.05 versus UV group at indicated time point.

FIG. 10A. Molecular structures of the four breakdown products of carnosol post-UVB.

FIG. 10B. The carnosol was irradiated with UVB first in the DMSO solution, and then a Mass Spectrum was run to detect the decomposition of the compound.

FIG. 10C. Four major decomposed compounds were detected, with their molecular structure and weight solved.

FIG. 11. The effect of carnosol or its decomposed molecules on cell proliferation was determined. The carnosol has some effect in inhibiting cell proliferation in normal skin cells, while the decomposed compound showed less toxicity. Both carnosol and its decomposed molecules showed partial protection of the UVB-irradiated cells.

FIG. 12. The effect of carnosol or its decomposed molecules on cell viability. Cell viability assay of carnosol and its UVB-decomposed molecules on normal skin cells in a time dependent manner (6, 12, and 24 h after UVB radiation). The data indicated that the decomposed molecule could increase cell viability, while both carnosol and the decomposed molecules can protect cell damage from UVB radiation in short term (within 12 h after treatment).

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this disclosure pertains.

### Examples

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

Material and Methods

Cell Culture.

Human keratinocyte HaCaT cells and mouse embryonic fibroblast (MEF) cells were grown in Dulbecco's Minimal Essential Medium (Cellgro) supplemented with 10% fetal bovine serum and penicillin/streptomycin, at 37° C. with 5% CO₂.

Uvb Radiation.

UVB was generated from a Bench XX-Series UV Lamp (UVP Inc.) equipped with two 15-watt UVB tubes (UVP Inc.). The intensity of UVB was calibrated by a UVP model UVX digital radiometer (UVP Inc.) after the lamps warmed up for 5 min. For irradiating cells, the dose rate for 8 mJ/cm² or 50 mJ/cm² of UVB radiation was 0.8 or 3.8 mW/s respectively. Medium was removed before exposing the cells to UVB. After UVB radiation, fresh medium was added to the culture plates with or without drugs, and the cells were continue incubating at 37° C. with 5% CO₂ until further analysis. Carnosol was disovled in DMSO and was irradiated with 50 mJ/cm² of UVB radiation at a dose rate of 3.8 mW/s before subjecting to cells or structure analysis using Mass Spectrum.

Drug Treatments.

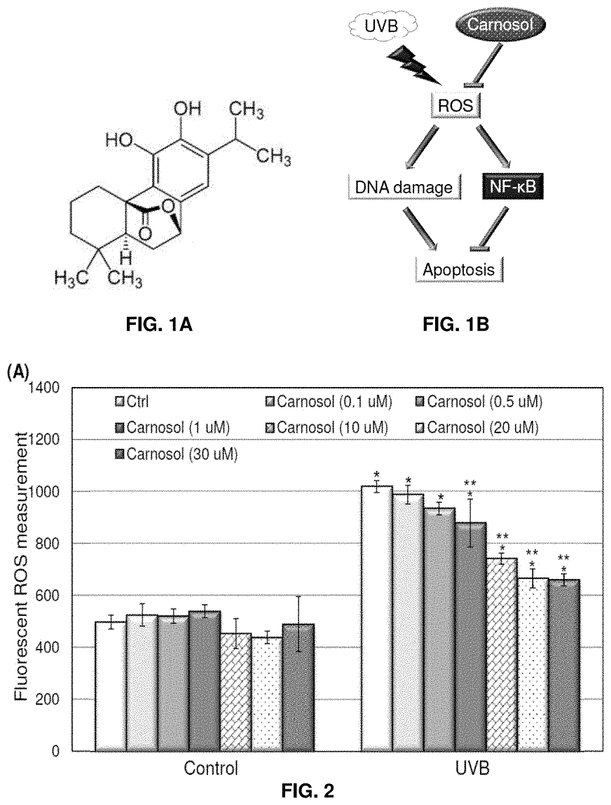

Carnosol (Cayman) (FIG. 1) was added to cells at indicated concentration at 1 h before exposing the cells to UVB radiation. After radiation, cells were continuously incubated with or without carnosol in the medium until further analysis.

ROS Measurement.

CM-H₂DCFDA (Invitrogen) was used to measure the total ROS level in cells. CM-H₂DCFDA was dissolved in DMSO to a stock solution of 500 μM and diluted in PBS to final concentration of 5 μM. CM-H₂DCFDA was added into cells 1 h prior to UVB exposure and the reading of fluorescence dye was recorded every 20 minutes using luminometer (Molecular devices Spectra Max M2).

Nitric Oxide and Peroxynitrite Measurement.

The level of NO* was measured by 5 mM diaminofluorescein diacetate (DAF-2DA) (ex490/em520 nm) and ONOO⁻ was measured by 5 mM diaminorhodamine (DAR) (ex488/em515 nm). 1×10⁵ HaCaT cells were seeded in 96-well plate one day before measurement. At indicated time point after UVB radiation and treatment with or without carnosol, medium was replaced with 200 μL reaction buffer, containing 10 μL L-arginine (1 mM) and 0.1 μL dye. The cells were then incubated in dark for 2 h at room temperature and the fluorescence was measured by luminometer (Molecular devices Spectra Max M2). The reading was then normalized by AlamarBlue cell viability assay (Life technology). 10% of the Alamar Blue reagent was added to cells with medium followed by 2 h incubation at 37° C. in dark then read the fluorescent at ex570/em590 nm using luminometer (Molecular devices Spectra Max M2).

Western Blot Analysis.

Cells were lysed with Nonidet P-40 (NP-40) lysis buffer (2% NP-40, 80 mM NaCl, 100 mM Tris-HCl pH 8.0, 0.1% SDS) with proteinase inhibitor mixture (Complete™, Roche Molecular Biochemicals) at indicated time point. Cell lysate was incubated on ice for 15 min and then centrifuged at 14,000 rpm at 4° C. for 15 min. Protein concentration was measured by Protein DC Assay kit (Bio-Rad Laboratories). Equal amounts of protein were subjected on SDS-PAGE and transferred to a nitrocellulose membrane. The membrane was then blocked in 5% milk in Tris buffered saline plus Tween 20 ® (TBST) for 45 min and probed with anti-yH2AX (Cell Signaling), anti-pCHK (Cell Signaling), anti-p276-NF-κB (Santa Cruz), anti-NF-κB p65 (Santa Cruz), anti-IκB (Santa Cruz), or anti-β-actin (Santa Cruz) at 4° C. overnight. After washing with TBST, the membrane was incubated with corresponding HRP-conjugated anti-rabbit or anti-mouse antibody for 45 min at room temperature. Membrane was then washed three times in TBST followed by two times in TBS, and developed in West Pico Supersignal chemiluminescent substrate (Pierce).

Electrophoretic Mobility Shift Assay.

A 22-bp synthetic oligonucleotide (5′-AGTTGAGGGGACTTTCCCAGGC-3′) [SEQ ID NO:1] containing the specific NF-κB-binding site was annealed and labeled with γ-³²P-ATP using T4 polynucleotide kinase. A DNA-binding reaction mixture of total 20 μL containing poly(dI:dC), labeled probe, binding buffer (10 mM pH 8.0 Tris HCl, 150 mM KCl, 0.5 mM EDTA, 0.1% Triton-X 100, 12.5% Glycerol and 0.2 mM DTT) and 10 jag of cell nuclear extract was incubated at room temperature for 30 min and loaded onto a 5% nondenaturing polyacrylamide gel for electrophoresis. The gel was run in 0.5×TBE buffer at 120 V, transferred to a double layer of Whatman paper and dried on a gel dryer for 45-60 mM at 76° C. The dried gel was used to expose an autoradiography film (Denville) at −80° C. and the NF-κB bound ³²P-labeled DNA was detected, and the band intensity was analyzed by Image J.

Cell Survival Analysis.

Total cell number of 1×10⁵ was used for each analysis using flow cytometer. Fluorescein isothiocyanate (FITC) conjugated-annexin V (ANXS)/propidium iodide (PI) apoptosis detection kit (BD Biosciences) were used to stain the cells through the determination of the loss of membrane phospholipid symmetry and membrane integrity. Cell survival rate (R) was calculated as: R=[1×10⁵−number of positive stained cells]/1×10⁵. Briefly, the cells were harvested by 0.25% trypsin digestion, combined with the cells floating in the medium and washed twice with cold PBS. The cells were then suspended in ANXS binding buffer (10 mM Hepes/NaOH, pH 7.4, 140 mM NaCl and 2.5 mM CaCl₂). The cell suspension was mixed with 5 μL ANXS-FITC and 5 μL PI. The cell mixture was incubated for 15 mM in dark at room temperature and the ANXS/PI double-stained cells were analyzed using a FACSort Flow Cytometer (BD Science) equipped with CellQuest software (BD Science).

Clonogenic Assay.

Immediate after treatment, cells were harvested with 0.25% trypsin-EDTA and counted, and then 5×10³ cells were plated in 6-well plates. After 6 days, cells were fixed with cold methanol for 10 mM at 20° C. and then stained by 1% crystal violet in 25% methanol for 10 mM. Cells were then rinsed with distilled water and colonies with a size greater than 0.4 mm were counted by Kodak IS in vivo F system equipped with Kodak Molecular Imaging Software (Eastman Kodak).

Results

Carnosol Reduces UVB-Induced ROS in Human Keratinocytes

The effect of different doses of carnosol on intracellular ROS level in HaCaT cells with or without UVB irradiation is demonstrated. The data show that 0.1 μM to 30 μM carnosol treatment alone had no statistic significant effect on the ROS level (FIG. 2, Control); and UVB (50 mJ/cm²) alone induced the ROS level by 2.1-fold at 6 h after the irradiation. The same treatment of carnosol reduced UVB-induced ROS level in a dose-dependent manner. The ROS level post-UVB was not statistically significantly changed with 0.1 μM or 0.5 μM carnosol treatment, while it was decreased to about 1.5-fold with 10 μM carnosol treatment and 1.3-fold with 20 or 30 μM carnosol treatment (FIG. 2. UVB). Since 30 μM carnosol showed no further deduction on ROS level compared to 20 μM, 20 μM carnosol treatment was used for further analysis.

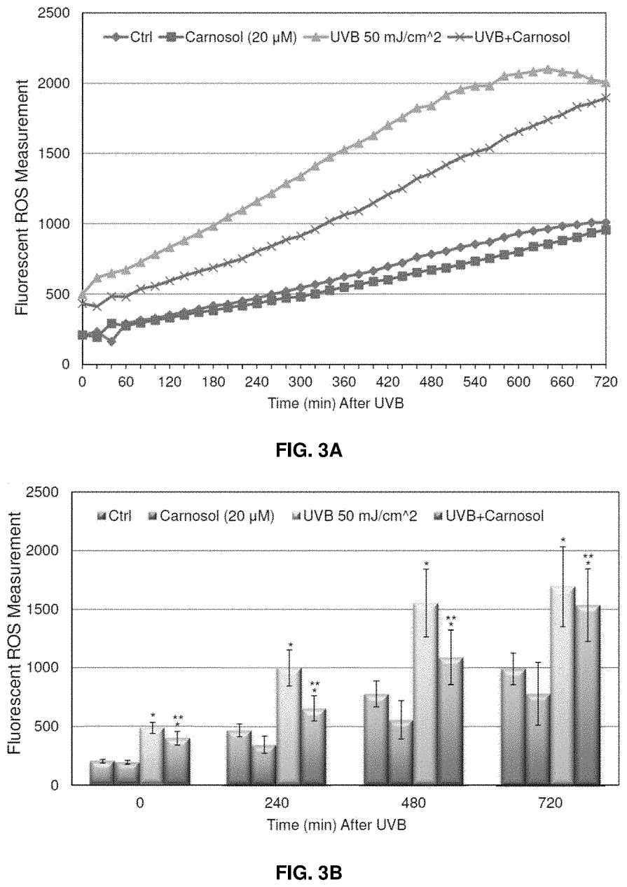

After selecting the dose of carnosol, determined the extent of effect of carnosol on UVB-induced ROS elevation in a time dependent manner was determined. The ROS level at 20 min interval for up to 12 h post UVB radiation. The data show that carnosol treatment did not statistically significant affect the ROS level in untreated cells, but carnosol continuously reduces the ROS level in the irradiated cells from 20 mM to 10 h post-UVB (FIG. 3A). Taking a closer reading, carnosol decreased the ROS level from 20% immediately after UVB treatment to approximate 30% at 240, 480, and 720 mM after the radiation (FIG. 3B). These results show that carnosol selectively inhibits the induction of some ROS induced by UVB radiation.

Both NO* and ONOO⁻ are induced by UVB radiation. By using DAF-2DA (5 mM) and DAR (5 mM), the effect of carnosol on intracellular NO* and ONOO⁻ level, in a time dependent manner upon UVB radiation, was also measured.

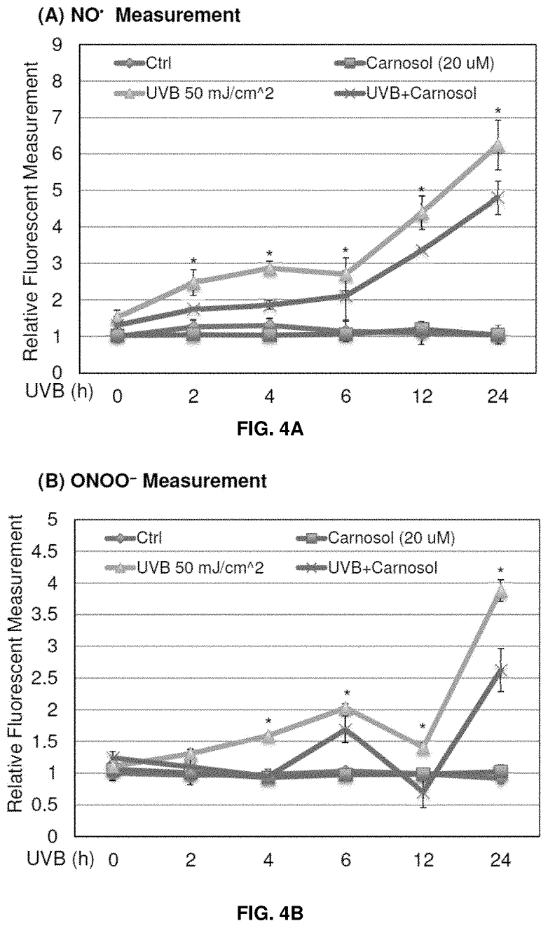

Both NO* and ONOO⁻ levels are increased upon UV radiation. With carnosol treatment, the data show that while carnosol had little effect on the NO; or ONOO⁻ levels of cells without radiation, carnosol reduces both NO* and ONOO⁻ level on average of about 40% from 2 h to 24 h post UVB radiation (FIG. 4).

Carnosol Protects Cells from UVB-Induced DNA Damage

The free radicals induced by UVB radiation is known to be able to cause indirect DNA damage in skin cells, and the accumulation of DNA damage will cause cancer development. As shown herein, carnosol reduces the UVB-induced ROS elevation; further carnosol protects cells from UVB-caused DNA damage.

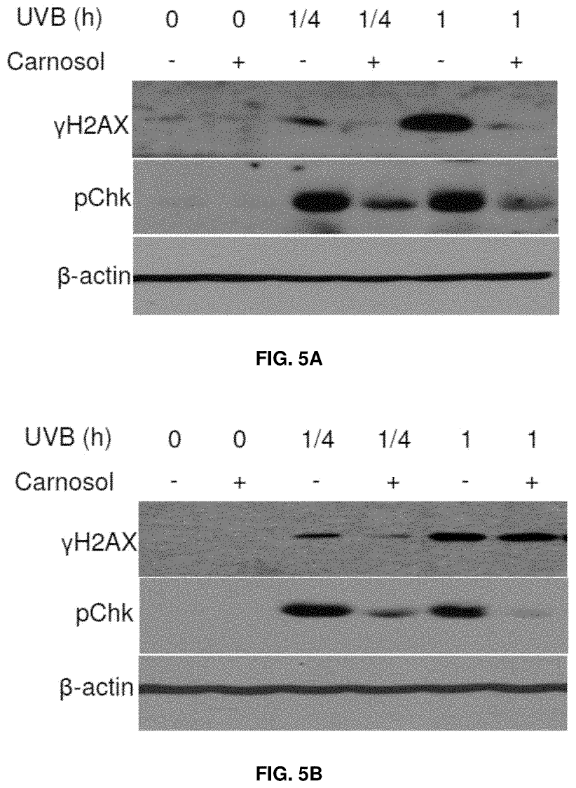

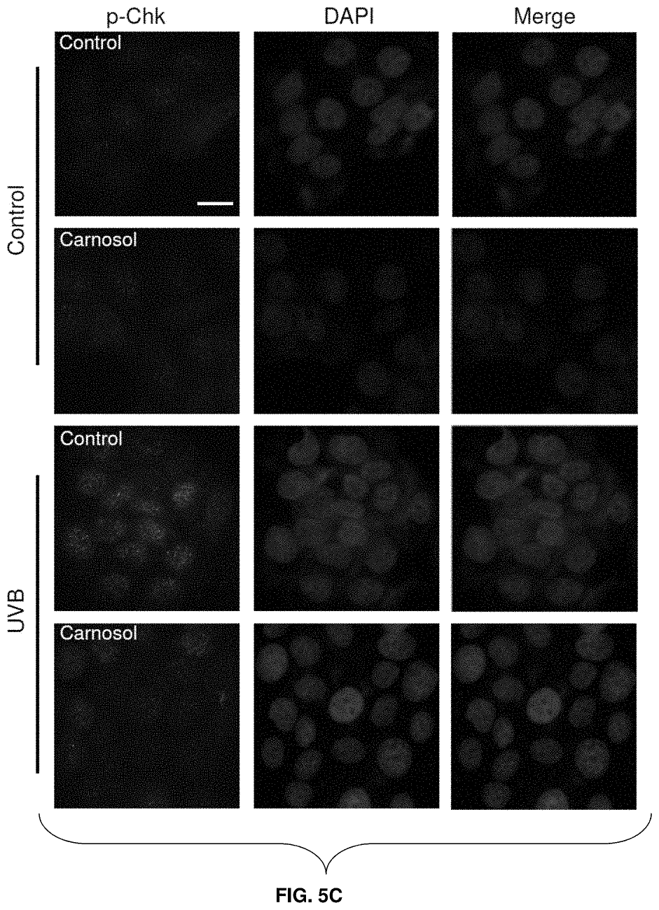

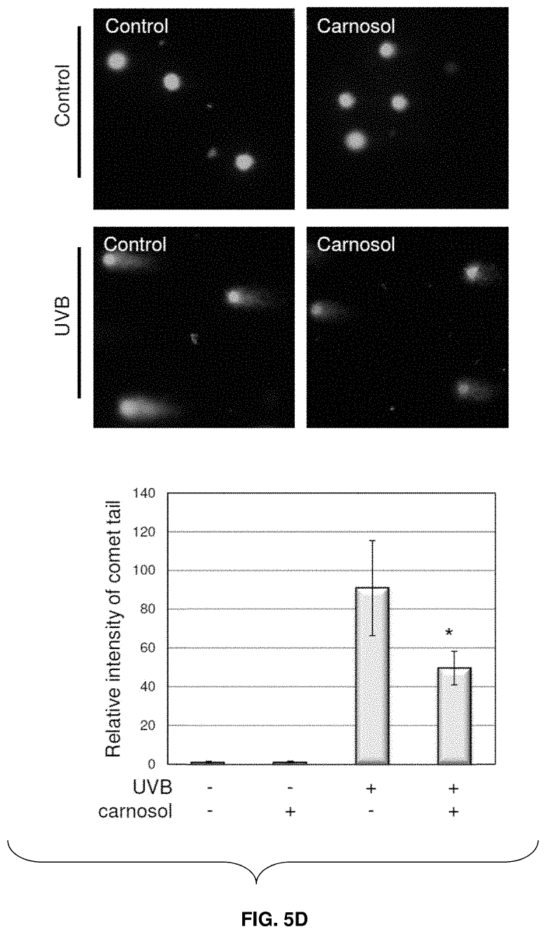

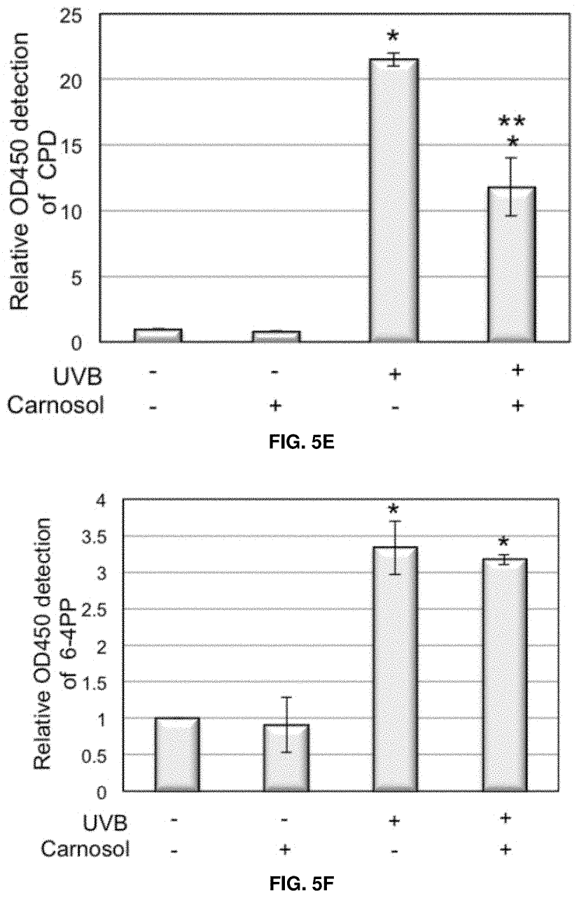

Both human keratinocyte (HaCaT) and mouse embryonic fibroblast (MEF) cells were used, and the protein levels of γH2AX and phosphor-Chk were used as DNA damage marker. In HaCaT cells, at early phase (15 mM and 1 h) post UVB radiation, the phosphorylation level of both H2AX and Chk was significantly reduced by carnosol treatment (FIG. 5A). Similar results are observed in MEF cells as both the phosphorylation level of H2AX and Chk reduced at early stage post UVB radiation (FIG. 5B). Similar results were confirmed by immunofluorescent assays which demonstrates that UVB radiation induces the expression level of p-Chk in nucleus, while carnosol treatment could significantly reduce the induction level (FIG. 5C). To further show the protection effect of carnosol on UVB-irradiated cell, a comet assay was performed. The data show that carnosol reduces the comet tail intensity almost by half upon UVB radiation (FIG. 5D). Thus, carnosol ast least partially protect the DNA damage from UVB radiation.

There are two major forms of DNA damage caused by UVB radiation: one is the direct DNA damage which forms CPD; the other is the oxidation-caused DNA damage, which causes the formation of 6-4 PP. In order to identify which form of DNA damage is protected by carnosol, a quantitative assay to identify the production of both DNA damage products after UVB radiation with or without carnosol treatment was performed. The data demonstrate that the formation of CPD was reduced by carnosol almost by half, while there is no significant reduction of 6-4 PP formation (FIG. 5E). The data show that carnosol protects cells from UVB-induced DNA damage by directly absorbing UVB energy (rather than reducing the oxidative stress on DNA damage, as previously believed).

Carnosol Protects UVB-Induced Cell Death

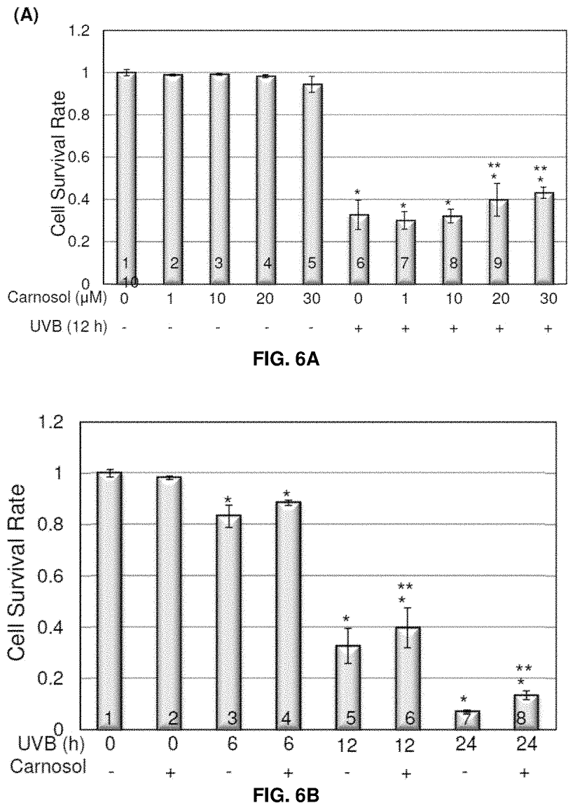

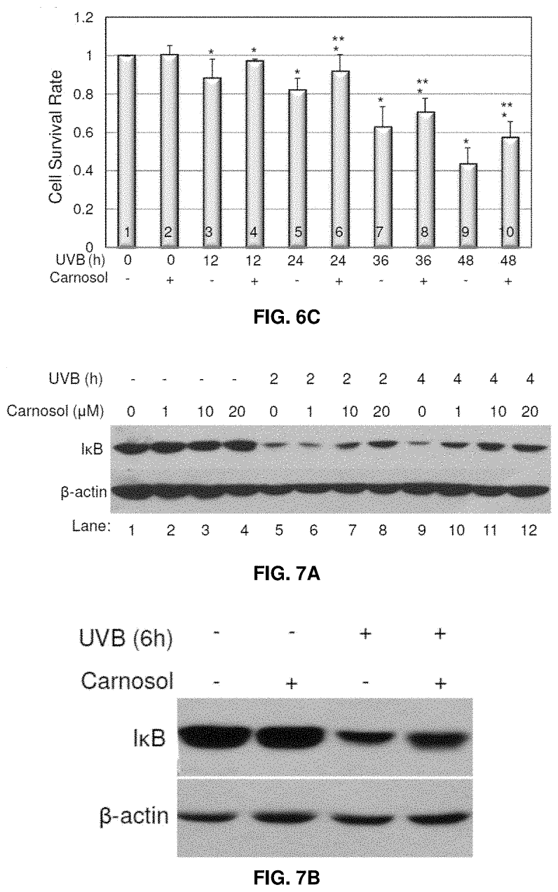

As carnosol reduced intracellular ROS level and protected cells from UVB-induced DNA damage, it was then determined whether carnosol protects cell death upon UVB radiation. Both time and dose dependent assays were performed using HaCaT cells. In dose dependent assays (1-30 μM), there was no significant cell survival change with carnosol treatment alone (FIG. 6A, Lanes 2-5 vs. 1). With UVB radiation, carnosol had no statistically significant effect at 1 or 10 μM (FIG. 6A Lanes 7, 8 vs. 6); while at 20 μM carnosol treatment, cell survival rate was increased from 20% of UVB radiation alone to 30% (FIG. 6A Lane 9 vs. 5), with no further increasing at 30 μM carnosol treatment while there was limited toxicity of carnosol to the cells without UVB radiation (FIG. 6A Lane 10 vs. 5).

For the time dependent cell survival rate assay, at 6 h post UVB radiation, there was no significance with or without 20 μM carnosol treatment (FIG. 6B Lane 4 vs. 3); while at 12 h post UVB radiation, cell survival rate increased from 30% to 40% (FIG. 6B Lane 6 vs. 5), and at 24 h post UVB radiation, cell survival rate increased from 7% to 15% (FIG. 6B Lane 8 vs. 7), which shows that the protection of cell lasts at least up to 24 h post UVB radiation (FIG. 6B).

Similar results were also observed in MEF cells: the cell survival rate was increased from about 88% to 97% at 12 h (FIG. 6C Lane 4 vs. 3), 80% to 90% at 24 h (FIG. 6C Lane 6 vs. 5), 62% to 70% at 36 h (FIG. 6C Lane 8 vs. 7), 43% to 58% at 48 h, respectively (FIG. 6C Lane 10 vs. 9). Taken together, the data show that carnosol protects cells from UVB-induced cell death, mediated by protection of DNA damage and reduction of elevated ROS level.

Carnosol Inhibits UVB-Induced NF-κB Activity

As ROS may mediate the induction NF-κB activity in UVB radiation, and carnosol reduces UVB-induced ROS level in cells, it was determined whether carnosol also affects the activity of NF-κB. The effect of carnosol on IκB protein level were determined in a time and dose dependent manner.

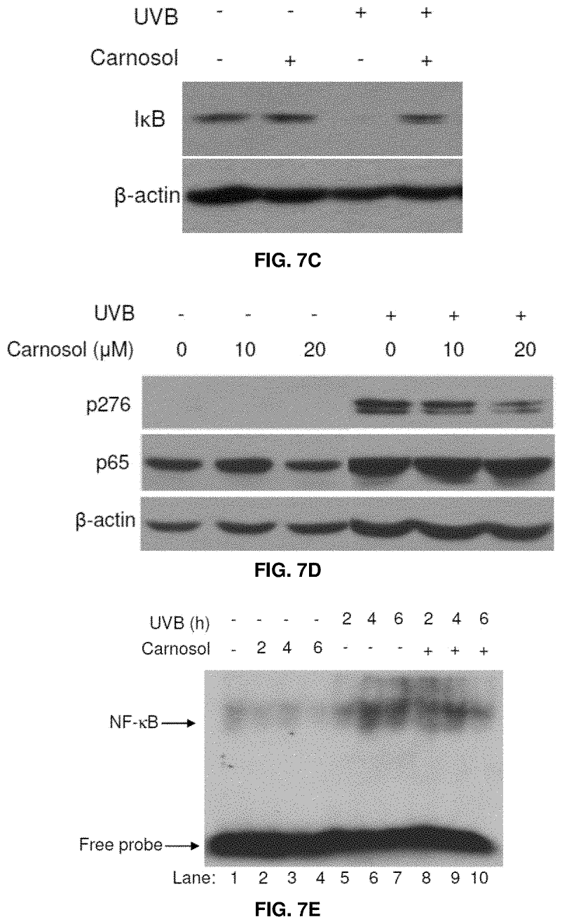

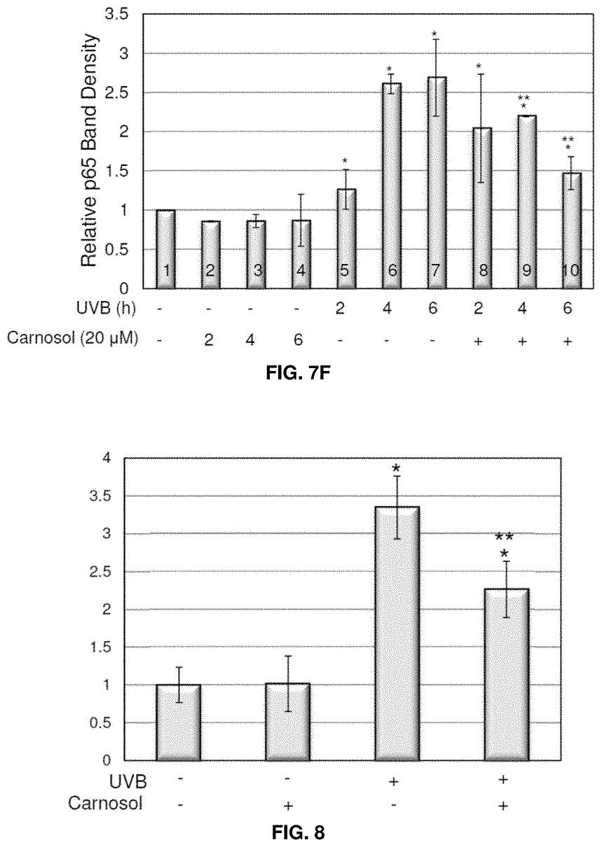

The data show that IκB level was decreased upon UVB radiation alone (FIG. 7A Lanes 9, 5 vs. 1), and carnosol protects IκB level at 2, 4 h post UVB radiation in a dose dependent manner (1, 10, 20 μM) (FIG. 7A Lanes 6-8 vs. 5; Lanes 10-12 vs. 9); and further, the protection exists at least until 6 h (FIG. 7B) in HaCaT cell. Similar results were also observed in MEF cells with the partial recovery of IκB level when the cells were treated with carnosol (20 μM) (FIG. 7C). As the phosphorylation of NF-κB usually indicates its activity, the phosphorylation of NF-κB at Serine 276 (S276) site was detected. Correspondingly, the data also show that carnosol reduces the phosphorylation of NF-κB at 5276 at 20 μM concentration (FIG. 7D).

To further confirm the NF-κB activity, EMSA assay was performed to test the binding activity of NF-κB to its DNA target. Upon UVB radiation, NF-κB activity induced by 20% at 2 h (FIG. 7E Lane 5 vs. 1), and peaked at about 2.6 fold at 4 and 6 h (FIG. 7E Lanes 6, 7 vs. 1). With the treatment of carnosol (20 μM), there was no statistically significant change at 2 h (FIG. 7E Lane 8 vs.5); however, the NF-κB activity induction was significantly reduced at 4 and 6 h by reducing to 2.2 and 1.5 fold respectively (FIG. 7E Lanes 9, 10 vs. 5.6). Altogether, the data show that carnosol inhibits NF-κB activity Rather than through the reduction of ROS level).

Carnosol Inhibits Normal Keratinocyte Transformation Upon UVB Radiation

UVB is a known carcinogen, as repeated exposure of normal cells to low dose UVB radiation can lead the normal cells to become cancerous cells.

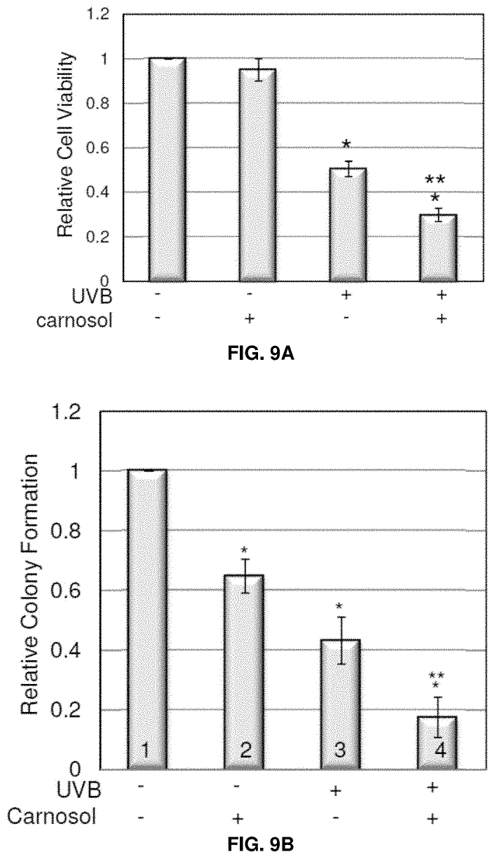

HaCaT cells were exposed to 10 mJ/cm² UVB radiation every 48 h for 14 days. As only cancerous cells can survive in the soft-agar, by staining the survival cells with fluorescent dye, the cell transformation rate was determined. As shown in FIG. 8, UVB radiation alone could induce the cell transformation to up to about 3.5 fold, while treat the cells with carnosol could reduce it to about 2.2 fold. This reduction shows that carnosol protects normal cells to transform to cancerous cell upon UVB radiation.

Carnosol Inhibits Cancer Cell Progression with a Synergetic Effect Upon UVB Radiation

Since carnosol protects cell death upon UVB radiation, how carnosol affects skin cancer cells was also determined. A431 human squamous carcinoma cell was used as it is one of the cancer types that keratinocyte would develop. Cell viability was determined by MTT assay. The data show that UVB alone could reduce the cell viability to about 50% in 24 h, compared to no treatment control; while treating the cells with carnosol further reduces the cell viability to 30%.

In addition, clonogenic assay was conducted to determine the progression rate of A431 cancer cell upon UVB radiation with or without carnosol treatment. With carnosol treatment alone, the colony formation rate decreased to about 65% (FIG. 9B Lane 2 vs. 1), while UVB radiation alone decreased the colony formation to about 43% (FIG. 9B Lane 3 vs. 1). With combined treatment of carnosol and UVB radiation, the colony formation was further decreased to about 17%. The data show that double treatment with carnosol and UVB radiation together leads to a synergistic effect on reduction of cancer cell survival (FIG. 9B).

Breaks Down Products of Carnosol after UVB Irradiation are Functional in Protecting Cells from UVB-Induces Growth Suppression and Death.

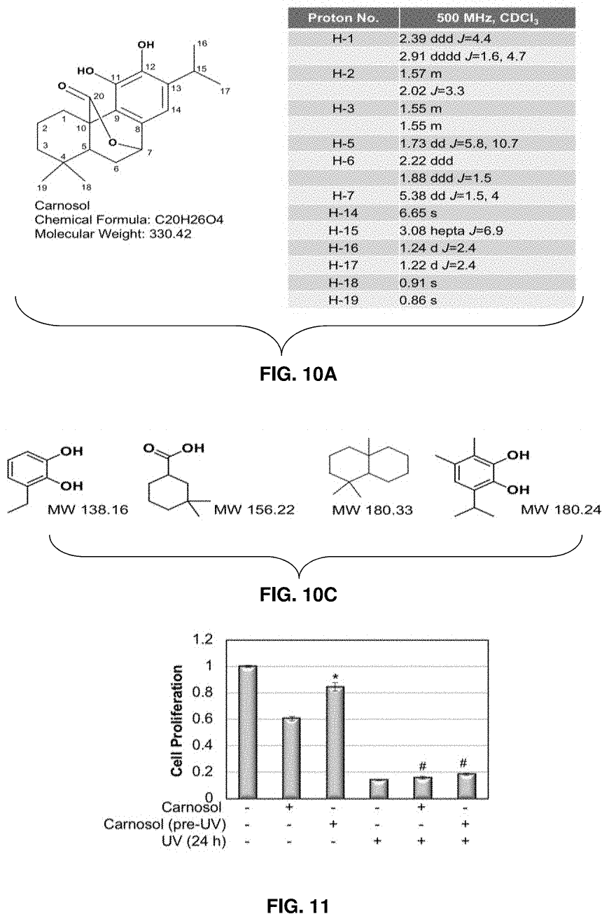

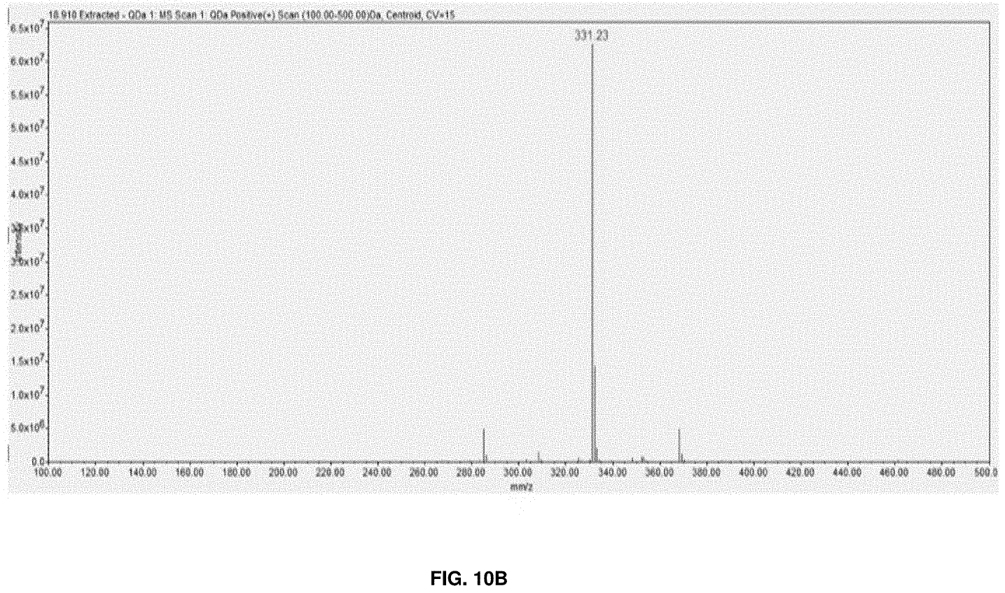

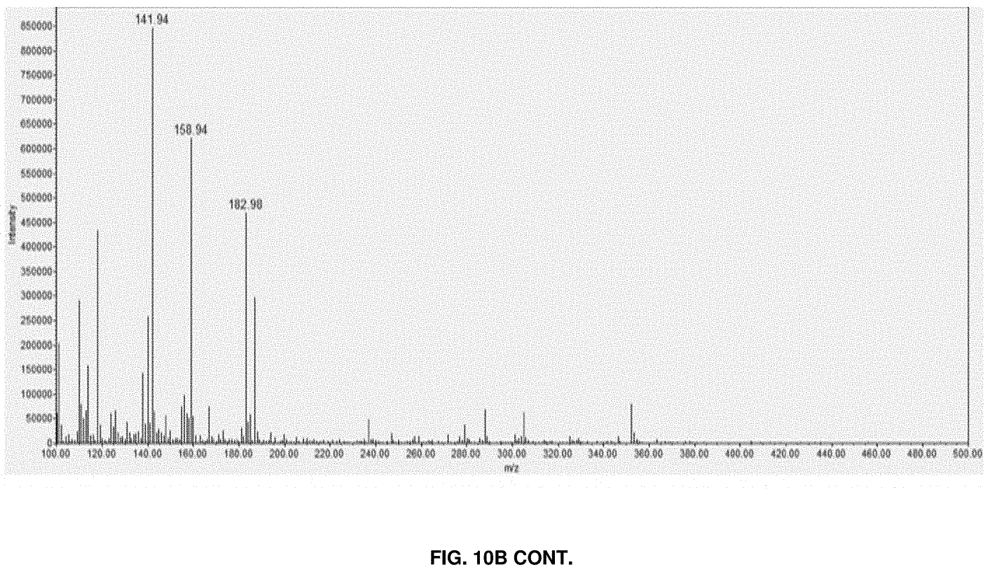

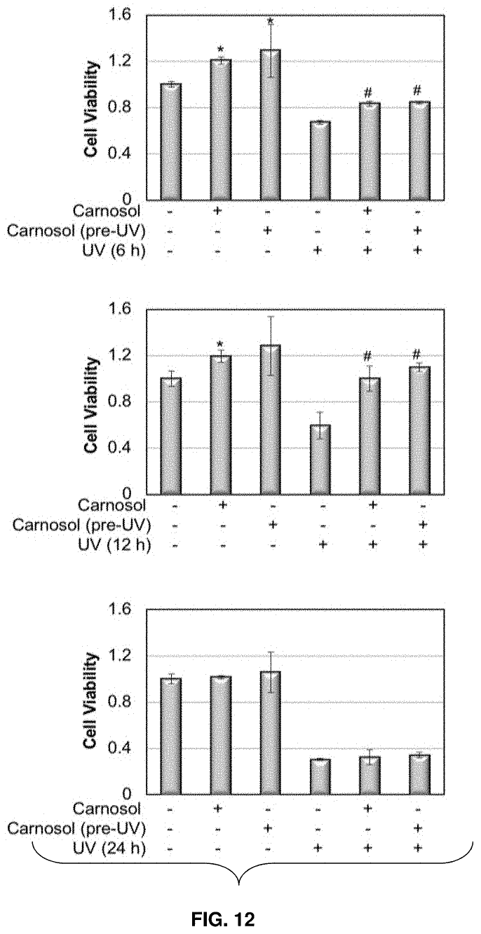

Carnosol absorbs UVB radiation and breaks down to 4 major products (FIG. 10). These four molecules, combined together at least, have the same affectiveness as carnosol in partially protecting cells from UVB-induced growth suppression and cell death (FIGS. 11-12), with less inhibitory effect than carnosol on cell growth (FIG. 11).

Additives and/or Alternatives to Carnosol

In certain embodiments, rosemary extract and/or rosemary powder can be added to carnosol. Carnosol is one of the rosemarinic acids in rosemary plant. When carnosol is used as a chemoprevention or therapy for UV-induced skin cancer, it is now believed that the whole rosemary extract and rosemary powder will have the similar or even higher activity as carnosol does. Rosemary extract or powder may be more attractive to some consumers because they are total “natural products”. Cost of making the extract or powder may also be less than the purified or synthetic compound (carnosol). One exemplary dosage is use as dietary supplement in about 30-60 mg rosemarinic acid. or 500-1000 mg rosemary extract per day.

Pharmaceutical Compositions

A pharmaceutical composition as described herein may be formulated with any pharmaceutically acceptable excipients, diluents, or carriers. A composition disclosed herein may comprise different types of carriers depending on whether it is to be administered in solid, liquid, or aerosol form, and whether it needs to be sterile for such routes of administration as injection. Compositions disclosed herein can be administered in a suitable manner, including, but not limited to topically (i.e., transdermal), subcutaneously, by localized perfusion bathing target cells directly, via a lavage, in creams, in lipid compositions (e.g., liposomes), formulated as elixirs or solutions for convenient topical administration, formulated as sustained release dosage forms, or by other method or any combination of the forgoing as would be known to one of ordinary skill in the art (see, for example, Remington's Pharmaceutical Sciences, 2003, incorporated herein by reference).

The compositions provided herein are useful for treating animals, such as humans. A method of treating a human patient according to the present disclosure includes the administration of a composition, as described herein.

The phrases “pharmaceutical” or “pharmacologically acceptable” refer to molecular entities and compositions that produce no adverse, allergic, or other untoward reaction when administered to an animal, such as, for example, a human. A carrier or diluent may be a solid, semi-solid, or liquid material which serves as a vehicle, excipient, or medium for the active therapeutic substance. Some examples of the diluents or carriers which may be employed in the pharmaceutical compositions of the present disclosure are lactose, dextrose, sucrose, sorbitol, mannitol, propylene glycol, liquid paraffin, white soft paraffin, kaolin, fumed silicon dioxide, microcrystalline cellulose, calcium silicate, silica, polyvinylpyrrolidone, cetostearyl alcohol, starch, modified starches, gum acacia, calcium phosphate, cocoa butter, ethoxylated esters, oil of theobroma, arachis oil, alginates, tragacanth, gelatin, syrup, methyl cellulose, polyoxyethylene sorbitan monolaurate, ethyl lactate, methyl and propyl hydroxybenzoate, sorbitan trioleate, sorbitan sesquioleate and oleyl alcohol, and propellants such as trichloromonofluoromethane, dichlorodifluoromethane, and dichlorotetrafluoroethane.

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols, and mixtures thereof, and in oils. Under ordinary conditions of storage and use, these preparations contain a preservative to prevent the growth of microorganisms. The pharmaceutical forms suitable for use include sterile aqueous solutions or dispersions and sterile powders for the extemporaneous preparation of sterile solutions or dispersions. In certain cases the form should be sterile and should be fluid to the extent that easy injectability exists. It should be stable under the conditions of manufacture and storage and may optionally be preserved against the contaminating action of microorganisms, such as bacteria and fungi. The carrier can be a solvent or dispersion medium containing, for example, water, ethanol, a polyol (i.e., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, such as, but not limited to, sugars or sodium chloride.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream, and petrolatum as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

It is further envisioned the compositions disclosed herein may be delivered via an aerosol. The term aerosol refers to a colloidal system of finely divided solid or liquid particles dispersed in a liquefied or pressurized gas propellant. The typical aerosol comprises a suspension of active ingredients in liquid propellant or a mixture of liquid propellant and a suitable solvent. Suitable propellants include hydrocarbons and hydrocarbon ethers. Suitable containers can vary according to the pressure requirements of the propellant. Administration of the aerosol can vary according to subject's age, weight, and the severity and response of the symptoms.

Dosage

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and the route of administration. Depending upon the dosage and the route of administration, the number of administrations of a preferred dosage and/or an effective amount may vary according to the response of the subject. The compounds of the present disclosure are generally effective over a wide dosage range. The practitioner responsible for administration can, in any event, determine the concentration of active ingredient(s) in a composition and appropriate dose(s) for the individual subject.

Naturally, the amount of active compound(s) in each therapeutically useful composition may be prepared in such a way that a suitable dosage can be obtained in any given unit dose of the compound. Factors such as solubility, bioavailability, biological half-life, route of administration, product shelf life, as well as other pharmacological considerations can be contemplated by those preparing such pharmaceutical formulations, and as such, a variety of dosages and treatment regimens may be desirable.

One exemplary dosage is 0.1 μM to 0.3 μM. Other dosages can range from 1 to 10 to 20 μM.

The dosages can depend on many factors, and can in any event be determined by a suitable practitioner. Therefore, the dosages described herein are not intended to be limiting.

In some embodiments, the compositions further include an additional active ingredient. The preparation of a pharmaceutical composition that contains at least one compound or additional active ingredient can be known to those of skill in the art in light of the present disclosure, as exemplified by Remington's Pharmaceutical Sciences, 2003, incorporated herein by reference. Moreover, for animal (e.g., human) administration, it can be understood that preparations should meet sterility, pyrogenicity, and general safety and purity standards as required by the FDA Office of Biological Standards.

Packaging of the Composition

After formulation, the composition is packaged in a manner suitable for delivery and use by an end user. In one embodiment, the composition is placed into an appropriate dispenser and shipped to the end user. Examples of final container may include a pump bottle, squeeze bottle, jar, tube, capsule or vial.

The compositions and methods described herein can be embodied as parts of a kit or kits. A non-limiting example of such a kit comprises the ingredients for preparing a composition, where the containers may or may not be present in a combined configuration. In certain embodiments, the kits further comprise a means for administering the composition, such as a topical applicator, or a syringe. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive, CD-ROM, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A composition, comprising an effective amount of carnosol as an active ingredient to reduce UVB-induced reactive oxygen species (ROS) levels in a cell, and to protect the cell from UVB-caused DNA damage; and, a pharmaceutically acceptable excipient, diluent, or carrier.

2. The composition of claim 1, wherein the carnosol is present in an amount effective to inhibit DNA damage caused by exposure to UVB irradiation.

3. The composition of claim 1, wherein the carnosol is present in an amount effective to inhibit skin cancer cell growth.

4. The composition of claim 1, wherein the carnosol is present in an amount effective to protect the cell from UVB-induced transformation.

5. The composition of claim 1, wherein the cell is a skin cell.

6. The composition of claim 1, wherein the carnosol is present in an amount effective to decrease cancer cell progression.

7. The composition of claim 5, wherein the cancer cell is a squamous carcinoma cell.

8. The composition of claim 1, wherein the composition is a cosmetic or dermatological composition for coating skin cells of a subject.

9. The composition of claim 1, wherein the composition is in the form of a spray, mist, aerosol, lotion, cream, solution, oil, gel, ointment, paste, emulsion or suspension.

10. A method of treating a cell, comprising: administering an effective amount of carnosol to reduce UVB-induced reactive oxygen species (ROS) levels in the cell, and to protect the cell from UVB-caused DNA damage.

11. The method of claim 10, wherein carnosol is administered in an amount effective to effective to inhibit DNA damage caused by exposure to UVB irradiation.

12. The method of claim 10, wherein the carnosol is present in an amount effective to inhibit skin cancer cell growth.

13. The method of claim 10, wherein the carnosol is present in an amount effective to protect the cell from UVB-induced transformation.

14. The method of claim 10, wherein the cell is a skin cell.

15. The method of claim 10, wherein the carnosol is present in an amount effective to decrease cancer cell progression.

16. The method of claim 15, wherein the cancer cell is a squamous carcinoma cell.

17. The method of claim 10, comprising topically applying carnosol to the cell.

18. The method of claim 17, wherein the topical composition is in the form of a spray, mist, aerosol, lotion, cream, solution, oil, gel, ointment, paste, emulsion or suspension.


SEQUENCE LISTING (1 sequence)

SEQ ID NO: 1 - DNA, 22 bp, Artificial Sequence
  Description of Artificial Sequence Synthetic oligonucleotide
agttgaggggactttcccaggc